(19) United States
(12) Patent Application Publication
(10) Pub. No.: US 2013/0101571 A9
(43) Pub. Date: Apr. 25, 2013

(54) ANTIDOTES FOR NITROBENZODIAZEPINES

(71) Applicants: Andrew H-J. Wang, (US); Shiuan-Woei Lin Wu, (US); Fu-Chuo Peng, (US); Che-An Wu, (US)

(72) Inventors: Andrew H-J. Wang, (US); Shiuan-Woei Lin Wu, (US); Fu-Chuo Peng, (US); Che-An Wu, (US)

(73) Assignee: Academia Sinica, Taipei (TW)

(21) Appl. No.: 13/428,232

(22) Filed: Mar. 23, 2012

Related U.S. Application Data
(60) Provisional application No. 61/467,160, filed on Mar. 24, 2011.

(51) Int. Cl. A61K 38/44; C12P 17/10; C12N 9/06; A61P 39/02

(52) U.S. Cl. 424/94.4; 435/121; 435/191

(57) ABSTRACT

Methods and compositions for detoxifying nitrobenzodiazepines with nitroreductase mutants.

### RELATED APPLICATION

This application claims the benefit of U.S. provisional application No. 61/467,160, filed Mar. 24, 2011 under 35 U.S.C. §119, the entire content of which is herein incorporated by reference.

### BACKGROUND OF THE INVENTION

Nitrobenzodiazepines (NBDZs) are a group of clinical sedative-hypnotic drugs that bind to a specific site on the gamma-amino butyric acid (GABA_{A}) receptor and enhance the inhibitory effect of GABA_{A} receptor on the central nervous system (CNS). NBDZ drugs are commonly used in treatment of insomnia, anxiety, muscle tension, and convulsions.¹

Flunitrazepam (FZ) is a NBDZ drug that has been used worldwide since the 1970s. Due to its quick-acting and long-lasting efficacy, FZ has become a favorable NBDZ-detoxifying agent for used in clinical remedy.² Despite the legitimate medical uses, there are a number of serious problems associated with the use of FZ. First, FZ is often cited as a “date rape” drug due to its high potency and its ability to cause amnesia during the course of action.³ Second, FZ is known to be addictive. Long-term abusive use of FZ leads to psychological and physical dependence and withdrawal syndrome once use is discontinued.⁴ Moreover, high concentrations of FZ can cause delirium, hallucinations and seizures, as well as global toxicity and death.⁴⁻⁶

In humans, metabolism of FZ occurs mainly in liver and involves oxidative and reductive reactions on the 1,4-benzodiazepine ring in FZ, yielding some unfavorable products. For example, N-desmethylflunitrazepam (DMFZ) and 3-hydroxyflunitrazepam (3OHFZ) are produced from demethylation and hydroxylation of FZ by hepatic cytochrome P450 2C19 and 3A4 (CYP2C19 and CYP3A4). DMFZ shows more prolonged hypnotic effect than FZ and could cause severe respiratory defects or even death via interaction with other co-administered drugs.^{7,8} Additionally, nitroso and hydroxyamine intermediates can be produced in the reduction reaction mediated by CYP3A4. These intermediates are reactive to cellular targets, resulting in hepatotxicity.⁹ On the other hand, FZ can also be degraded to 7-aminoflunitrazepam (7AFZ) via the reduction reaction catalyzed by NADPH-cytochrome P450 reductase (P450R). 7AFZ is the major FZ metabolite in urine and is harmless to the body.¹⁰

### SUMMARY OF THE INVENTION

The present invention is based on the unexpected discovery that a number of E. coli type I nitroreductase (NfsB) mutants, e.g., N71S/F124W, are highly effective in converting FZ to inactive metabolite 7AFZ under both aerobic and anaerobic conditions. These mutants therefore are effective in detoxifying NBDZs.

Accordingly, one aspect of the present disclosure relates to a method for detoxifying a NBDZ drug (e.g., flunitrazepam, nitrazepam, cloazepam, nimetazepam, and meclonazepam). This method includes administering to a subject in need thereof (e.g., a human patient suffering from or suspected of having nitrobenzodiazepine poisoning) an effective amount of a nitroreductase mutant (e.g., a mutant of a bacterial type I nitroreductase), which, as compared to its wild-type counterpart, comprises a mutation at one or more positions corresponding to 40, 41, 68, 70, 71, and 124 in SEQ ID NO:1. In one example, the nitroreductase mutant contains G at a position corresponding to position 40 in SEQ ID NO:1, L at a position corresponding to position 41 in SEQ ID NO:1, G at a position corresponding to position 68 in SEQ ID NO:1, H, W, or Y at a position corresponding to position 70 in SEQ ID NO:1, S at a position corresponding to position 71 in SEQ ID NO:1, and/or H, K, N, W, or Y at a position corresponding to position 124 in SEQ ID NO:1. In another example, the nitroreductase mutant is a mutant of E. coli type I nitroreductase NfsB having the amino acid sequence of SEQ ID NO: 1. The E. coli NfsB mutants to be used in the methods described herein include, but are not limited to, S40G, T41L, Y68G, F70H, F70W, F70Y, N71S, F124H, F124K, F124N, F124W, F124Y, and N71S/F124W. The subject to be treated by the methods described herein can be co-administered (e.g., simultaneously or sequentially) with one or more of the nitroreductase mutants described herein and a NBDZ drug.

In another aspect, the present disclosure relates to a method for converting a NBDZ drug to a 7-amino-NBDZ, comprising contacting the NBDZ drug with one or more of the nitroreductase mutants described herein in an amount sufficient to convert the NBDZ to 7-amino-NBDZ. The contacting step can be performed by administering an effective amount of the one or more nitroreductase mutants to a subject in need of the treatment.

In yet another aspect, the present disclosure relates to kits for treating anxiety, insomnia, or convulsion, and/or relaxing muscles. The kits comprise one or more NBDZ drugs and one or more of the nitroreductase mutants described herein. Optionally, the just-noted kits can further comprise Flumazenil.

Also within the scope of this disclosure are (i) a pharmaceutical composition for use in detoxifying a NBDZ, the composition comprising one or more of the nitroreductase mutants described herein, a pharmaceutically acceptable carrier, and optionally a NBDZ drug or Flumazenil, and (ii) use of the pharmaceutical composition in manufacturing a medicament for NBDZ detoxification.

The details of one or more embodiments of the invention are set forth in the description below. Other features or advantages of the present invention will be apparent from the following drawings, detailed description of several embodiments, and also from the appended claims.

### BRIEF DESCRIPTION OF THE DRAWINGS

The drawings are first described.

FIG. 1 is a diagram showing the nitroreduction process of Flunitrazepam (FZ).

FIG. 2 is a diagram showing the design of NfsB mutants having higher activity in converting FZ to 7AFZ based on resolved crystal structure of the enzyme. Panel A: a crystal structure with active site pockets identified by hotspot analysis.²⁵ Panel B: a chart showing the activity of wild-type and mutants of NfsB enzymes in converting FZ to 7AFZ. Each value represents the mean±S.D. from three incubations. The mean value of 7AFZ formation of wild-type NfsB was 51.85±2.19 ng. The asterisk (*) indicates a significant difference (P<0.05). The top panel shows protein purity as determined by SDS-PAGE.

FIG. 3 is a diagram showing structural and functional characterization of the N71S/F124W mutant. Panel A: surface representations of active site pockets in wild-type NfsB and the NfsB mutants of N71S, F124W and N71S/F124W. Panel B: values of pocket volumes and mouth areas in the active sites of NfsB proteins.²⁶ Panel C: convertion of FZ to 7AFZ as catalyzed by the purified NfsB enzymes under anaerobic and aerobic conditions. Each value represents the mean±S.D. from three incubations. The mean values of 7AFZ formation of wild-type NfsB were 51.85±2.19 ng (anaerobic) and 29.52±6.6 ng (aerobic), respectively. Asterisks (*, **) indicate a significant difference (P<0.05).

FIG. 4 is a chart showing the inhibitory effect of FZ on E. coli cells transformed with wild-type or mutant NfsB as determined by a disk diffusion assay. LB plates were uniformly streaked with 100 μl of pET46 vector (A), wild-type (B) or N71S/F124W (C) transformed E. coli BL21 (DE3). ∘: FZ;; : metronidazole (MTZ); ▾: a combination of FZ and MTZ.

FIG. 5 is a diagram showing the in vivo antidote activity of double mutant N71S/F124W. Panel A: a chart showing variation of sleep-time in miceafter treatment with NfsB. Each value represents the mean±S.D. from four mice. The mean value of the control group was 273.75±24.96 min Asterisk (*) indicates significant difference (P<0.05). Panel B: a chart showing in vivo NfsB serum half-life.

FIG. 6 is a diagram illustrating conformational changes of the N71S/F124W mutant. Panel A: a stereo view of the active site of wild-type NfsB. Hydrogen bonds are indicated with dotted lines. Panel B: a stereo view of the active site of the N71S/F124W mutant. Hydrogen bonds are indicated with dotted lines. Panel C: a profile of major changes in the active site pockets of wild-type NfsB (left panel) and the N71S/F124W mutant (right panel). Arrows indicate the positions in the N71S/F124W mutant where the major movements occurred.

FIG. 7 is a diagram showing the complex structure of the N71S/F124W mutant interacted with nicotinamide. Panel A: a stereo view of the structural comparison between the N71S/F124W/nicotinamide complex and the unbounded mutant. B: a stereo view of the hydrophobic sandwich effects in the wild-type (WT) enzyme, the WT-nicotinate complex (WT-NIC; PDB: 1ICR), and the N71S/F124W-nicotinamide complex(NF-NIA), indicating the role of residue 124 in coordination with FMN for ligand binding.

FIG. 8 is a diagram illustrating a schematic model of NfsB active site changes, leading to enhanced substrate accessibility.

### DETAILED DESCRIPTION OF THE INVENTION

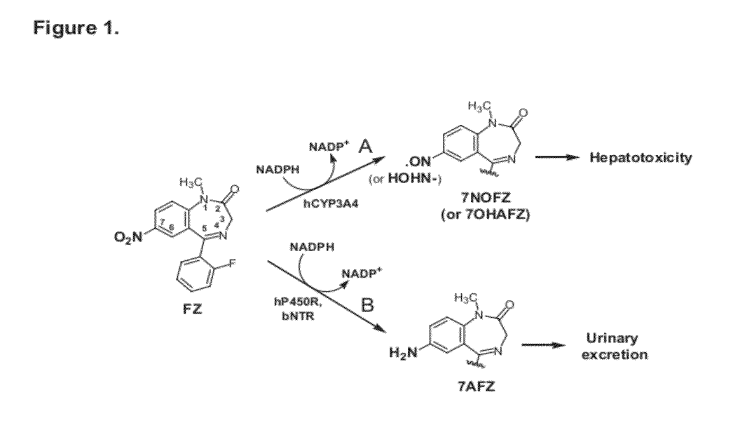

As noted above, in vivo metabolism of NBDZs produces various metabolites, some of which are toxic and/or active, while others are nontoxic and inactive. FIG. 1 shows the metabolism pathway of Flunitrazepam (FZ). Several enzymes have been characterized as FZ reductases in the body. These enzymes use NADPH as an electron donor for catalysis. As shown in route A, hepatic cytochrome P450 3A4 (hCYP3A4) converts FZ to reactive metabolite 7-nitrosoflunitrazepam (7NOFZ) or 7-hydroxyaminoflunitrazepam (7OHAFZ). Both 7NOFZ and 7OHAFZ exhibit hepatotoxicity. Route B shows that both hepatic NADPH-cytochrome P450 reductase (hP450R) and intestinal bacterial nitroreductase (bNTR) catalyze FZ to form 7-aminoflunitrazepam (7AFZ), which is the main urinary metabolite of FZ and has low toxicity.

Flumazenil, a competitive antagonist of NBDZ, is currently the first choice in clinical uses for treating NBDZ poisoning. Flumazenil treatment has several disadvantages. First, repeated or continuous infusion of flumazenil may be required to prolong the effectiveness and prevent resedation in liver failure patients administered with high concentrations of NBDZ. Such patients often have high accumulation of flumazenil in the body, which may evoke seizures and breathing difficulties.¹¹ Second, in chronic users and those who have physical dependence on NBDZ, flumazenil may hasten withdrawal syndrome and seizures, and even cause convulsions in patients with brain injury.¹² Third, flumazenil is not effective at eliminating residual NBDZ or its active metabolites in the circulatory system. Such residual NBDZ or active metabolites may lead to severe outcomes, especially in patients who have taken high doses. It is of greatimportance to develop new methods for detoxifying NBDZ.

The present disclosure is based on the discovery that a number of E. coli type I nitroreductase (NfsB) mutants are unexpectedly effective in converting FZ to the inactive metabolite 7AFZ under both aerobic and anaerobic conditions. Thus, NfsB mutants are therapeutic agents for converting FZ and other nitrobenzodiazepine drugs, thereby detoxifying nitrobenzodiazepine drugs.

Nitroreductasesare a well-characterized family of enzymes that catalyze the reduction of nitro groups in a wide range of substrates to produce corresponding hydroxylamine compounds. Wild-type E. coli type I nitroreductase NfsB was found to be effective in converting FZ to 7AFZ, an inactive metabolite.^{6-10, and 13} However, the reductase activity of wild-type NfsB under aerobic conditions is usually lower than the enzymatic activity under anaerobic conditions. This may affect its utility as a biological scavenger inside the body.

Disclosed herein are a number of E. coli type I nitroreductase (NfsB) mutants that, unexpectedly, exhibited high activity in converting NBDZs to their inactive metabolite 7-amino-BDZs under both aerobic and anaerobic conditions. Accordingly, the present disclosure relates to uses of one or more nitroreductase mutants for converting NBDZs (e.g., at least 50%, 60%, 70%, 75%, 80%, 85%, 90%, 95%, 97%, or 99%) to their inactive 7-amino-BDZ metabolites, thereby detoxifying the NBDZ (e.g., curing, healing, alleviating, relieving, altering, remedying, ameliorating, improving, or affecting one or more side effects caused by a NBDZ or reducing or alleviating a subject's addiction to NBDZ).

The nitroreductase mutants to be used in the methods described herein can be derived from any wild-type nitroreductase, preferably a bacterial nitroreductase. Examples include, but are not limited to, E. coli type I nitroreductase NfsB (NP_{—}415110, SEQ ID NO:1 shown below), E. cloacae type I nitroreductase NR (Q01234), and S. typhimurium type I nitroreductase Cnr (AAO69891).

Amino Acid Sequence of E. coli type I nitroreductase NfsB (NP_{—}415110; SEQ ID NO:1)

| | |
|---|---|
| 1 | mdiisvalkr hstkafdask kltpeqaeqi ktllqyspss tnsqpwhfiv asteegkarv |
| 61 | aksaagnyvf nerkmldash vvvfcaktam ddvwlklvvd qedadgrfat peakaandkg |
| 121 | rkffadmhrk dlhddaewma kqvylnvgnf llgvaalgld avpiegfdaa ildaefglke |
| 181 | kgytslvvvp vghhsvedfn atlpksrlpq nitltev |

It was known in the art that other wild-type nitroreductases share high sequence identity to the E. coli NfsB enzyme, e.g., those described under GenBank Accession Numbers YP_{—}001742694, ZP_{—}08394123, ZP_{—}07680640, EGC08677, EHC75770, and YP_{—}003942755. Other nitroreductases can be retrieved from any gene database using the amino acid sequence of an available nitroreductase, e.g., SEQ ID NO:1, as a search query. Functional domains in a nitroreductase can be identified by comparing its amino acid sequence with amino acid sequences of other enzymes, e.g., the E. coli NfsB noted above. The crystal structure of this E. coli enzyme has been described herein, which provides sufficient information about the function/structure correlation of this enzyme. See the Example below. Using this NfsB enzyme as a reference, functional domains of another NfsB enzyme can be readily identified.

Compared to their wild-type counterparts, the nitroreductase mutants described herein include a mutation at one or more positions corresponding to 40, 41, 68, 70, 71, and 124 in SEQ ID NO:1 (see boldfaced residues in SEQ ID NO:1 above). Given the cross-species homology of this enzyme, these positions in another nitroreductase (e.g. an E. coli isoform of SEQ ID NO:1 or a non-E. coli NfsB) can be readily identified by aligning the amino acid sequence of the other nitroreductase with SEQ ID NO:1. In one example, the nitroreductase mutant contains G at a position corresponding to S40 in SEQ ID NO:1, L at a position corresponding to T41 in SEQ ID NO:1, G at a position corresponding to Y68 in SEQ ID NO:1, H, W, or Y at a position corresponding to F70 in SEQ ID NO:1, S at a position corresponding to N71 in SEQ ID NO:1, and/or H, K, N, W, or Y at a position corresponding to F124 in SEQ ID NO:1, as well as their conservative substitutes.

As known in the art, a “conservative amino acid substitution” can be an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The nitroreductase mutants can share at least 80% (e.g., 85%, 90%, 95%, 98%, 99%, or 99.5%) sequence identity to their wild-type counterparts. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Alternatively, the nitroreductase mutants can include only one or more (e.g., 1, 2, 3, 4, 5, 6, 7, 8, 9, 10, 15, or 20) amino acid residue substitutions or deletions as compared to their wild-type counterparts. In some embodiments, the mutants include only amino acid residue substitutions at one or more of the positions noted above.

Any of the nitroreductase mutants disclosed herein can be prepared by routine mutagenesis and recombinant technology. For example, the nucleotide sequence coding for a wild-type nitroreductase can be obtained and mutations introduced into one or more sites of interest via conventional mutagenesis techniques. The nucleotide sequence coding for a mutant nitroreductase can be inserted into an expression vector and introduced into a host cell for expression.

The nitroreductase mutant thus prepared can be mixed with a pharmaceutically acceptable carrier to form a pharmaceutical composition. A pharmaceutically acceptable carrier is compatible with the active ingredient(s) in the composition (and preferably, capable of stabilizing it) and not deleterious to the subject to be treated. For example, solubilizing agents such as cyclodextrins, which form more soluble complexes with a nitroreductase mutant, or more solubilizing agents, can be utilized as pharmaceutical carriers for delivery of the enzyme mutants. Examples of other carriers include colloidal silicon dioxide, magnesium stearate, sodium lauryl sulfate, and D&C Yellow #10. See, e.g., Remington's Pharmaceutical Sciences, Edition 16, Mack Publishing Co., Easton, Pa. (1980); and Goodman and Gilman's “The Pharmacological Basis of Therapeutics”, Tenth Edition, Gilman, J. Hardman and L. Limbird, eds., McGraw-Hill Press, 155-173, 2001.

The pharmaceutical composition mentioned above, containing an effective amount of one or more nitroreductase mutants can be administered to a subject in need of the treatment via a suitable route, e.g., oral, parenteral, by inhalation spray, topical, rectal, nasal, buccal, vaginal, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as TWEENs or SPANS or other similar emulsifying agents or bioavailability enhancers which are commonly used in the manufacture of pharmaceutically acceptable solid, liquid, or other dosage forms can also be used for the purposes of formulation. See, e.g., Remington's Pharmaceutical Sciences, 16th edition, Mack Publishing Co., Easton, Pa. (1980); and Goodman and Gilman's “The Pharmacological Basis of Therapeutics”, Tenth Edition, Gilman, J. Hardman and L. Limbird, eds., McGraw-Hill Press, 155-173, 2001.

A composition for oral administration can be any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. The pharmaceutical composition described herein can also be administered in the form of suppositories for rectal administration.

“An effective amount” as used herein refers to the amount of a nitroreductase mutant that alone, or together with further doses or one or more other active agents, produces the desired response, e.g. alleviating a side effect caused by NBDZ or reducing reliance on NBDZ. This may involve only slowing the progression of the side effect temporarily, although more preferably, it involves halting the progression of the side effect permanently. This can be monitored by routine methods, such as physical examination and blood test.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The interrelationship of dosages between animals and humans (e.g., based on milligrams per meter squared of body surface or milligrams per body weight) is well known in the art. See, e.g., Freireich et al., (1966) Cancer Chemother Rep 50: 219. Body surface area may be approximately determined from height and weight of the patient.

Subjects who need treatment of the nitroreductase mutants described herein include those who are suffering from, suspected of having, or at risk for NBDZ poisoning. They can be human patients who require treatment or are under treatment of a NBDZ drug (e.g., flunitrazepam), or are addicted to the drug. When a subject who needs NBDZ treatment is likely to develop toxicity induced by the NBDZ drug (e.g., flunitrazepam, nitrazepam, cloazepam, nimetazepam, and meclonazepam), one or more of the nitroreductase mutants may be co-administered with the NBDZ drug to such a subject for preventive purposes. The one or more of the nitroreductase mutants can also be co-used with one or more additional detoxifying drugs, such as flumazenil. In one example, the one or more nitroreductase mutants are administered simultaneously with the NBDZ drug or the additional detoxifying drug(s), e.g., formulated in one pharmaceutical composition or formulated in separate compositions and mixed before use. In another example, they can be administered sequentially.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example

### Identification of NfsB Mutants Effective in Detoxifying NBDZs and Uses Thereof

The abbreviations used in this example include:
NBDZ: nitrobenzodiazepine;
FZ: flunitrazepam;
FMN: flavin mononucleotide;
7AFZ: 7-aminoflunitrazepam;
MTZ: metronidazole;
NIA: nicotinamide;
CNS: central nervous system;
GABA: gamma-amino butyric acid;
NADPH: nicotinamide adenine dinucleotide phosphate;
ELISA: enzyme-linked immunosorbent assay;
SDS-PAGE: sodium dodecyl sulfate polyacrylamide gel electrophoresis

NBDZs are a family of addictive drugs, which can cause severe neurological effects and even death. Bacterial type I nitroreductase NfsB (EC 1.5.1.34) has been reported to catalyze NBDZ compounds to produce an inactive 7-aminobenzodiazepine (7ABDZ) metabolite with promising activity. In this study, the crystal structure of the NfsB enzyme has been resolved, providing insight as to the structure/function correlation of this enzyme.

Based on this crystal structure, various NfsB mutants have been developed, which showed high activity in converting FZ, a commonly used NBDZ drug, to its 7-amino metabolite in vivo. In contrast to the wild-type enzyme, conformational changes were observed in the enzyme mutants at the active site of NfsB-N71S/F124W including the flipping of Trp¹²⁴ and Phe⁷⁰ side chains as well as the extended hydrogen bond network of Ser⁷¹. In addition, stacking sandwich attractions of Trp¹²⁴-nicotinamide-FMN were found in the complex structure of NfsB-N71S/F124W, implying the importance of Trp¹²⁴ in substrate accessibility. In the NfsB-N71S/F124W mutant, the active site pocket was significantly enlarged in contrast to that of the wild-type enzyme. The enlarged active site pocket may allow increased 7-aminoflunitrazepam (7AFZ) production under both aerobic and anaerobic conditions.

An E. coli disk diffusion assay indicated that nitroreduction of FZ via the NfsB-N71S/F124W mutant neither exhibited toxicity nor increased MTZ toxicity. A mouse anti-hypnosis study showed, compared to the vehicle group (274±25 min), a 50% decrease of sleeping time in the NfsB-N71S/F124W group (138±22 min), with over 50% of the enzymes still remaining in the mice sera after 24 hours. Taken together, results from this study demonstrated that NfsB-N71S/F124W, as well as other nitroreductase mutants, could be used as an effective therapeutic agent for FZ-induced hypnosis and provide the molecular basis for rational design of NfsB and the like in the future.

### Experimental Procedures

### Cloning, Expression, and Purification

The coding region of nitroreductase nfsB was amplified from genomic DNA of E. coli DH5α via PCR and cloned into the pET-46LIC vector (Novagen). All NfsB mutants were prepared using the QuickChange site-directed mutagenesis kit (Stratagene) and confirmed by DNA sequencing of the entire NfsB coding sequence. The validated constructs were subsequently transformed into E. coli BL21 (DE3) competent cells (Novagen) for protein expression, following the procedures described previously.¹³ Briefly, overnight culture (8 ml) of a single clone was inoculated into 800 ml of LB medium (BD) containing 100 μg/mL ampicillin. The cells were grown to 0D₆₀₀ 0.5-0.6 under 180 rpm shaking and induced with 1 mM isopropyl-β-thiogalactopyranoside (IPTG) at 20° C. After 20 h, the cells were harvested by centrifugation at 5000×g for 15 mM The resulting cell pellets were suspended in 50 mM Tris-HCl buffer (pH 7.5, containing 500 mM NaCl and 20 mM imidazole) and disrupted with a cell disrupter (Constant Systems) at 4° C. The cell-free extract was subsequently loaded onto a HiTrap Ni-NTA column (GE Healthcare). His-tagged NfsB recombinant proteins were eluted with a linear gradient of imidazole from 20 to 250 mM. The collected fractions were pooled and further purified via DEAE ion-exchange columns (GE Healthcare). The active fractions were dialyzed to 50 mM Tris-HCl buffer (pH 7.5, containing 50 mM NaCl) and stored at −80° C.

Purity of the enzymes thus produced was examined by SDS-PAGE.

### Enzyme Crystallization and Data Collection/Processing

Crystals of wild-type NfsB and the N71S/F124W mutant (8 mg/mL) were grown at room temperature in a buffer containing polyethylene glycol 350 monomethyl ether (PEG 350 MME, 37% w/v) and 100 mM MES, pH 6.0. Similarly, crystals of the NfsB-nicotinamide complex were obtained by adding 15 mM nicotinamide (dissolved in H₂O) in the same reservoir solution. By using sitting-drop vapor diffusion, the square-shaped crystals appeared within one week. In each case, crystals were rinsed with 40% PEG 350 MME solution (in 100 mM MES, pH 6.0) as a cryoprotectant for few seconds immediately prior to flash cooling to 100K. Diffraction data were collected at 100 K using beamline PF6A at KEK (native crystals) and BL13B at NSRRC (mutant and complex crystals). All data were indexed, integrated and scaled using HKL2000.¹⁵ Full data collection statistics are listed in Table 1.

TABLE 1
Data collection and refinement statistics for the NfsB crystals

| Crystal dataset | Wild type | N71S/F124W | N71S/F124W-NIA |
|---|---|---|---|
| Data collection | | | |
| Space group | P2₁ | P2₁ | P2₁ |
| Unit cell a, b, c (Å) | 56.4, 50.5, 77.6 | 47.4, 87.2, 53.8 | 47.2, 86.2, 53.5 |
| β (°) | 109.7 | 110.4 | 111.0 |
| Resolution (Å) | 30-1.65 (1.72-1.65) | 30-1.75 (1.81-1.75) | 30-1.85 (1.92-1.85) |
| No. observations | 181,415 | 224,896 | 202,945 |
| Unique reflections | 49,655 (4,883) | 39,297 (3,859) | 113,097 (32,529) |
| Completeness (%) | 99.6 (99.1) | 94.3 (92.9) | 95.9 (94.8) |
| Average I/σ (I) | 27.5 (7.5) | 34.3 (5.6) | 23.8 (5.8) |
| R_{merge} (%) | 4.6 (25.1) | 4.5 (27.5) | 4.2 (21.0) |
| Refinement | | | |
| No. reflections (F > 0) | 47,710 (4,411) | 38,149 (3,550) | 40,494 (4,481) |
| R_{work} (95% data) | 0.19 (0.23) | 0.20 (0.23) | 0.22 (0.25) |
| R_{free} (5% data) | 0.23 (0.28) | 0.22 (0.26) | 0.26 (0.29) |
| r.m.s.d. bond distance (Å) | 0.005 | 0.006 | 0.005 |
| r.m.s.d. bond angle (°) | 1.13 | 1.10 | 1.14 |
| Ramachandran plot (% residues) | | | |
| Most favored regions | 93.9 | 93.4 | 93.6 |
| Additional allowed regions | 5.6 | 6.1 | 5.9 |
| Generously allowed regions | 0.3 | 0.3 | 0.3 |
| Average B (Å²)/no. non-H atoms | | | |
| Protein | 15.5/3370 | 18.9/3372 | 17.0/3366 |
| Water | 24.7/382 | 27.2/306 | 26.3/140 |
| FMN | 49.0/62 | 53.6/62 | 50.8/62 |

Numbers in parentheses are for the highest resolution shells. All positive reflections were used in the refinement.

### Structure Determination and Model Refinement

The crystals thus obtained were isomorphous and in monoclinic forms as previously reported.^{16,17}. All crystal structures were determined by molecular replacement using CNS (Crystallography and NMR System).¹⁸ NfsB structure 1DS7.pdb (P4₁2₁2) was used as the starting structure to refine the wild-type structure of NfsB.¹⁹ NfsB-N71S/F124W and its nicotinamide complex structures were sequentially refined. Geometry restraint files for FMN and nicotinamide were taken from HIC-UP database.²⁰ Model adjustment and ligand fitting were done manually using the molecular graphics program XtalView using SinmaA-weighted Fₒ-F_{c} and 2Fₒ-F_{c} difference maps as a guide.²¹ Final refinement parameters are shown in Table 1. The atomic coordinates and structure factors of the various structures have been deposited in the Protein Data Bank. See Table 1.

### 7AFZ Production and Kinetic Study

Production of 7AFZ was performed under both aerobic or anaerobic conditions. For anaerobic incubation, nitrogen was inlet into an anaerobic incubator (Biotrace). The reaction mixture consisted of FZ (64 μM), purified NfsB proteins (10 μg), MgCl₂ (3.3 mM), and NADPH (2 mM) in 100 mM potassium phosphate buffer (pH 7.4). After incubation for 2 h at 37° C., the reaction was terminated by the addition of a methanol solution (200 μl) containing bromazepam (1 μg/mL), which acted as an internal control. The reaction products were extracted via sonication and analyzed by HPLC at 240 nm with a procedure described previously.¹³ The detection limit for 7AFZ or FZ was less than 3-5 ng with 83-86% of recovery.

A kinetic study was carried out by monitoring the rate of FZ disappearance. FZ (4-256 μM) was incubated with purified NfsB proteins (10 μg) under the aerobic conditions described above for 2 h in the presence of NADPH at a fixed concentration of 2 mM.

### Disk Assay for FZ Sensitivity

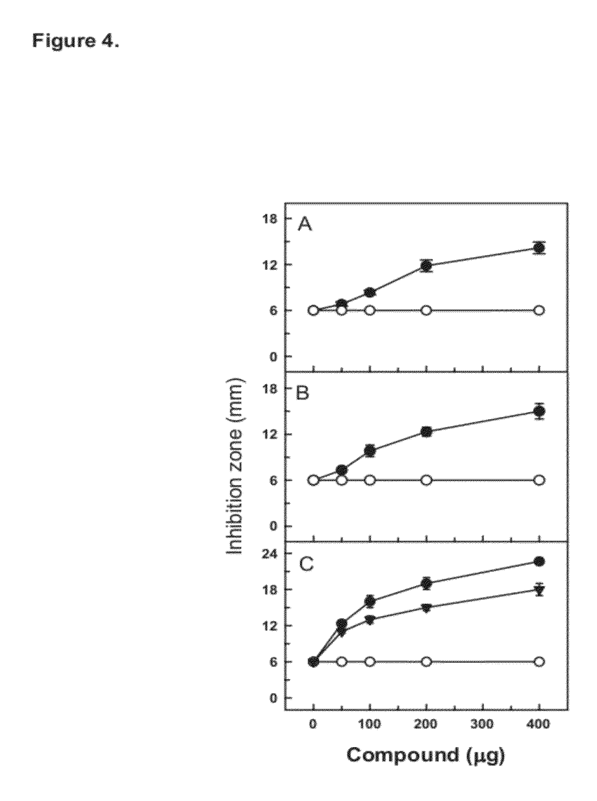

LB plates were uniformly streaked with 0.1 ml of bacterial cell suspensions having an OD₆₀₀ value of 0.8. Sterile 7 mm filter paper disks saturated with 7 μl of each chemical, including FZ alone, metronidazole (MTZ) alone, and a combination of FZ and MTZ at a ratio of 1/1 by weight, in the amounts indicated in FIG. 4 were placed on the plates. The cells were incubated for 24 hrs before the zones of inhibition were measured.²²

### Animal Study

Male Balb/c mice were purchased from the Animal Center, College of Medicine, National Taiwan University (NTUMC), and were housed in the air-conditioned animal quarters on a 12-h light/dark cycle (temperature 23-25° C.). The animals had free access to water and rodent laboratory chow (Purina Mills Inc.) with the experimental procedures being approved by the Institutional Care and Use Committee at NTUMC.

A sleep-time study in mice was started at the age of seven weeks (body weight ranged from 17.6-19.2 g) following experimental procedures modified from previous studies.²³ Briefly, mice were divided into three groups (at least 4 mice per group) and injected i.p. with 2 mg/kg body weight of FZ from the stock solution (0.4 mg/ml in corn oil). One minute later, mice in Group I, Group II, and Group III were injected i.v. with a vehicle control, 1 mg/ml of wild-type NfsB enzyme (dissolved in 100 mM potassium phosphate buffer, pH 7.4), and 1 mg/ml of the N71S/F124W mutant enzymes (also dissolved in 100 mM potassium phosphate buffer, pH 7.4), respectively, via the tail lateral veins of the FZ-treated mice. The ability of the treated mice to stand on a stainless steel laboratory holder was examined at various time points. A mouse was characterized as losing the reflex ability if it could not stand on the holder for 10 seconds; A mouse was defined as having regained the reflex ability if it could stand on the holder for at least 10 seconds. The time between losing and regaining the reflex ability was defined as sleep-time and recorded for each group.

### Direct Competitive ELISA Assay

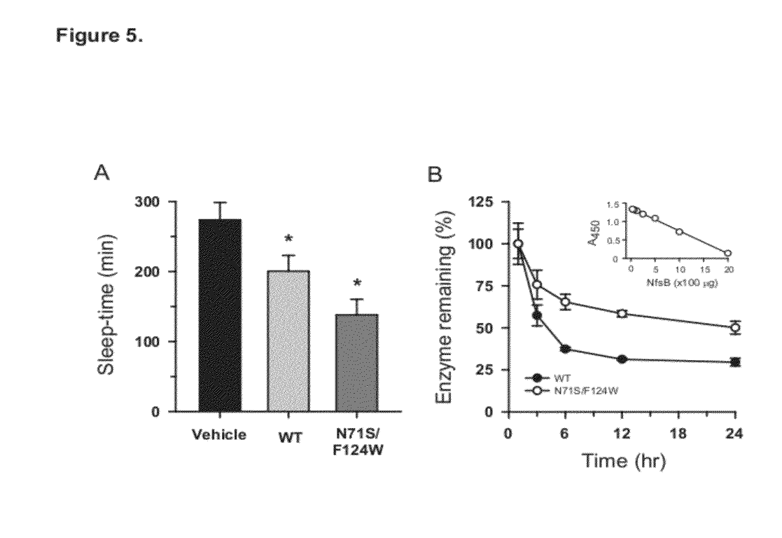

The ELISA assay used in this study was a modified version of a previously described method.²⁴ Briefly, 96-well microtitre plates were coated with 100 μL of anti-NfsB antibody solution (4 μg/ml in PBS; 13) overnight at 4° C., then washed with PBS containing 0.05% Tween 20 (PBST) in triplicate, then incubated with a blocking buffer containing PBST and 1% BSA. NfsB standards (containing 40 μl sera of untreated mice), or samples (containing 40 μl sera from treated mice collected 1, 3, 6, 12, and 24 hours after the treatment) were placed in the above-noted blocking buffer (60 μL) and added to the wells, which were incubated overnight at 4° C. Enzyme tracer solution (100 μl 6×His mAb/HRP, 1:10,000 in PBS) were added to the wells and incubated at 37° C. for 30 min Subsequently, the wells were washed and TMB substrate solution was added (100 μl). After incubation for 15 min at room temperature, a sulfuric acid solution (2N, 50 μl) was added to stop color development. The absorbance was measured at 450 nm. Standard curve (31.25 to 2000 pg of NfsB) was plotted as absorbance (A₄₅₀) vs. logarithm of NfsB concentration (see FIG. 5B). NfsB concentration in the sera samples of the treated groups were calculated based on the standard curve.

### Statistical Analysis

Data were presented as arithmetic mean±S.D. Statistical analysis was established with one-way ANOVA followed by Dunnett's post hoc test for the groups in comparison with the same control. P<0.05 was considered significant.

### Results

### NfsB Mutants Having Increased Activity to Convert FZ to 7AFZ

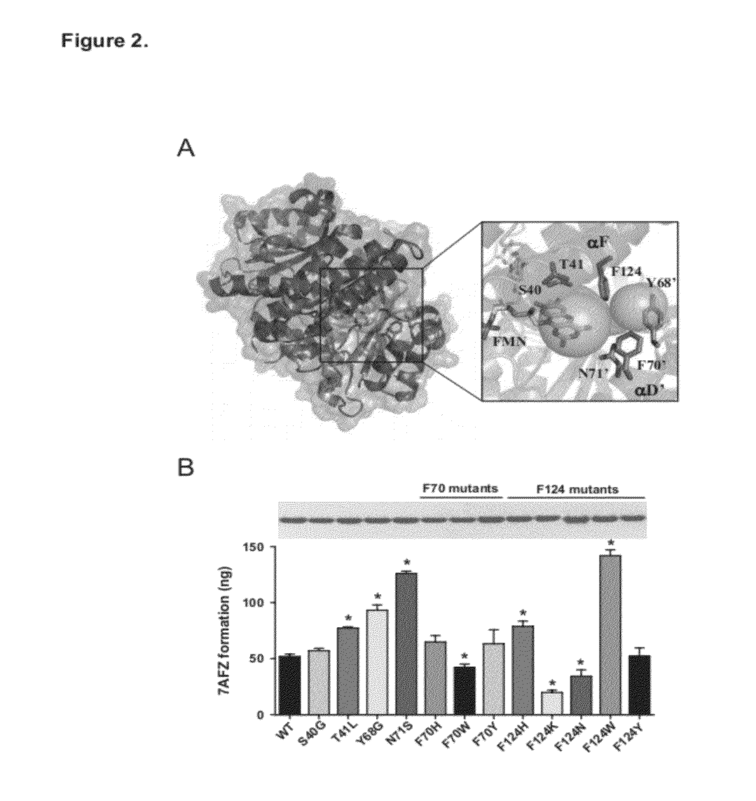

To investigate the overall architecture of NfsB for rational enzyme design, the crystal structure of wild-type NfsB was first solved in 1.65 Å (FIG. 2A, left panel). Applying HotSpot Wizard prediction (see ref. 25), a hypothetical sphere on top of FMN in the active site of NfsB was identified, which was surrounded by several residues (i.e. hotspots) capable of influencing the catalytic activity including Ser⁴⁰ and Thr⁴¹ (αB-β1 loop), Tyr⁶⁸ (αC-αD loop), Phe⁷⁰ and Asn⁷¹ (αD) and Phe¹²⁴ (αF) (FIG. 2A, right panel). These residues were subsequently chosen for initial protein engineering and the resulting twelve mutants (S40G, T41L, Y68G, N71S, F70H, F70W, F70Y, F124H, F124K, F124N, F124W and F124Y) were successfully produced in E. coli and purified with a purity higher than 95% as determined by SDS-PAGE (FIG. 2B).

The ability of the NfsB mutants to convert FA to 7AFZ was determined as described above. The amount of 7AFZ thus produced was measured by HPLC analysis. Under anaerobic conditions, production of 7AFZ was significantly increased when FZ was incubated with mutant T41L, Y68G, N71S, F124H, or F124W as compared to the wild-type enzyme. For example, the amounts of 7AFZ formed in the presence of N71S (126±2.06 ng) and F124W (141.88±5.28 ng) were, respectively, 2.4- and 2.7-fold higher than in the presence of the wild-type enzyme (51.85±2.19 ng) (FIG. 2B). Mutants S40G, F70H, F70Y and F124Y also showed higher activity in converting FA to 7AFZ as relative to the wild-type enzyme. FIG. 2B.

### Enlarged Active Site of NfsB Mutants

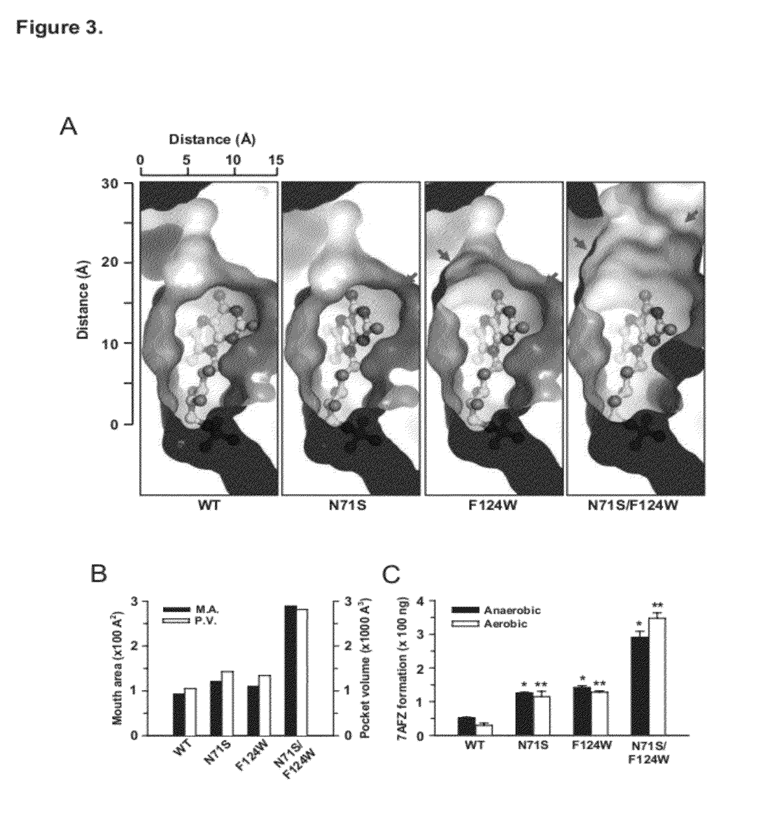

To examine the molecular profile of NfsB mutants having enhanced enzymatic activity, crystal structures of N71S and F124W were determined. Surface representation of the mutants was made and conformational changes near the active site were observed. A few angstroms shift around the active site pocket was observed (indicated by the arrows in FIG. 3A), indicating that the active site pockets in the mutants are larger than that in the wild-type enzyme. To further verify this result, the crystal structure of double mutant N71S/F124W was resolved. A significantly larger pocket was observed in this double mutant as compared to that in the wild-type enzyme. The major conformational changes occur adjacent to the ring for binding to FMN (FIG. 3A). Following the calculation method described in ref. 26, pocket volume and mouth area of the active site of the N71S/F124 double mutant were determined as 2.7 (2813 vs. 1052 Å³) and 3.1 (289 vs. 93 Å²) fold larger than those of the wild-type enzyme, respectively. These values of the double mutant are 2-fold higher relative to the corresponding values of the N71S or F124W mutant (FIG. 3B).

In addition, the formation of 7AFZ as catalyzed by NfsB-N71S/F124W was examined under both anaerobic and aerobic conditions. Surprisingly, the productions of 7AFZ in both conditions were 5.6 (290.52±18.38 vs. 51.85±2.19 ng in anaerobic) and 11.77 (347.56±16.59 vs. 29.52±6.6 ng in aerobic) fold greater than that of the wild-type enzyme, which showed relatively low activity in converting FZ to 7AFZ under aerobic conditions(FIG. 3C).

Kinetic Analysis and Sensitivity from FZ Reduction

The amounts of FZ were determined during the course of the reaction catalyzed by NfsB. Under aerobic conditions, the enzymatic reaction catalyzed by wild-type NfsB and the N71S/F124W mutant followed the Michaelis-Menten kinetics in the presence of NADPH and FZ. Their kinetic parameters were summarized in Table 2. The specific activity of the N71S/F124W mutant against FZ was 2.4-fold greater than that of the wild-type enzyme (195.5 vs. 82.9 nmol/min/mg protein). The Vₘₐₓ value of the mutant is 1.7-fold higher than that of the wild-type enzyme (3.13 vs. 1.89 μM/min).

TABLE 2
Specific activities and kinetic parameters of wild type
and N71S/F124W of NfsB

| | Specific activity | Kₘ | Vₘₐₓ |
|---|---|---|---|
| NfsB | nmol/min/mg protein | μM | μM/min |
| Wild type | 82.9 (1.0) | 71.42 | 1.89 |
| N71S/F124W | 195.5 (2.4) | 111.11 | 3.13 |

Data were analyzed and calculated via linear plot (13). The amounts of FZ were obtained via the standard nitroreductase assay of E. coli NfsB in the presence of a fixed concentration of cofactor NADPH (2 mM).

The in vivo FZ disk assay was performed as described above. The zones of FZ inhibition on nfsb-wt and nfsb-n71s/f124w-transformed E. coli BL21(DE3) were measured. Metronidazole, a known toxic nitroaromatic compound activated by nitroreductase, was used as a positive control. In contrast to the dose-dependent manners of metronidazole, FZ did not show any inhibitory effect on both E. coli cells expressing either the wild-type enzyme or the N71S/F124W mutant, even at a dosage as high as 400 μg (FIG. 4). This result indicates that the N71S/F124W mutant can effectively convert FA to non-toxic 7AFZ.

### In Vivo Potency of NfsB-N71S/F124W

To investigate the in vivo potency of NfsB, Balb/c mice were used for the anti-FZ hypnosis study. Prior to the injection of the wild-type and N71S/F124W mutant enzymes, mice were injected subcutaneously with 1 mg FZ. Both the wild-type enzyme and the N71S/F124W mutant significantly reduced the sleep-time in treated mice(FIG. 5A). Analysis of NfsB proteins in mice serum showed that at least 50% of N71S/F124W remained in the serum 24 hours after the injection, while only 30% of the wild-type enzyme was detected at the same time point (FIG. 5B).

### Structural Analysis of the N71S/F124W Mutant

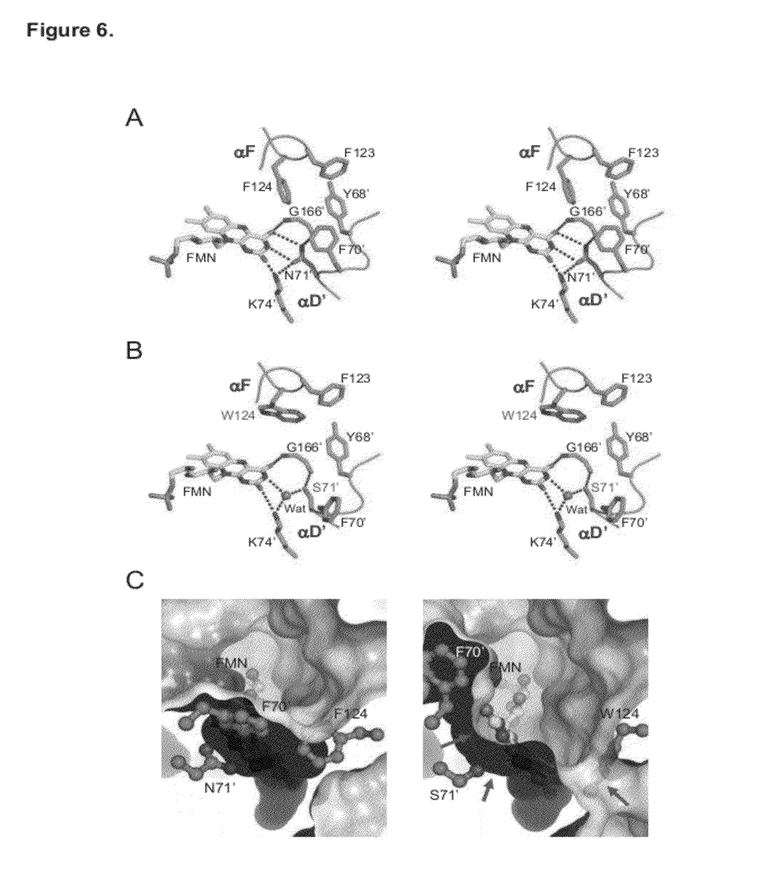

Superimposition of the backbone of N71S/F124W with that of the wild-type protein showed that the mutant has similar structures to the wild-type enzyme in general but has certain variations in the αD′, αF and αC′-αD′ loop, which constitute the entrance of the active site (FIG. 6A). A detailed view of the active site of the N71S/F124W mutant reveals three major alterations in amino residues as compared to the wild-type enzyme(FIG. 6B). First, the orientation of the Ser⁷¹ side chain had been changed in the mutant, resulting in the rearrangement of the hydrogen bonding network in relation to Lys⁷⁴, Gly¹⁶⁶ and FMN. This may be caused by the replacement of Asn⁷¹ OD1 with a bound water molecule, which mediates Ser⁷¹ OG, FMN N3 and Lys⁷⁴ NZ. Second, the side chain of Trp¹²⁴ had rotated away from the FMN in a direction toward the non-polar pocket consisting of Tyr⁶⁸, Phe⁷⁰, Phe¹²³ and Met¹²⁷. This change may reinforce the stacking force in the pocket and result in the slight movements of these residues. Third, the side chain of Phe⁷⁰ had rotated apparently in an opposite orientation, which could be seen in the homodimer of the N71S/F124W mutant with a stable and equal manner. The dramatic movement of the aromatic ring of Phe⁷⁰ may be attributed to the stacking effect by Trp¹²⁴ and the steric effect by Ser⁷¹.

Taken together, these changes had made the active site pocket of the N71S/F124W mutant larger than that of the wild-type enzyme. Ser⁷¹ and Trp¹²⁴ created more space nearby and above the isoalloxazine ring of the FMN individually, and Phe⁷⁰ extended the pocket opening (arrow 3, FIG. 6C).

### Ligand Binding of NfsB-N71S/F124W

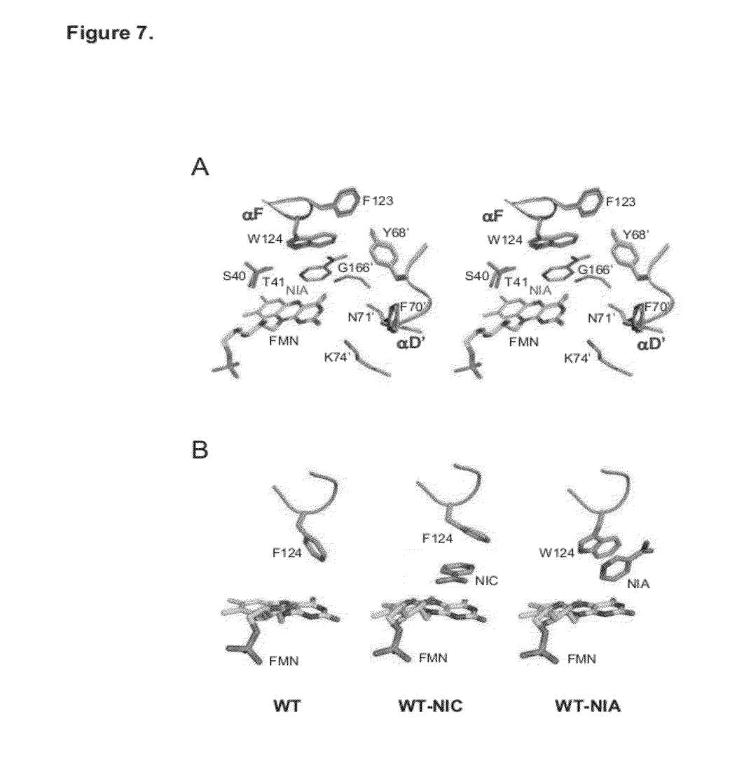

In order to investigate the ligand binding activity of NfsB mutants, the crystal structure of the N71S/F124W mutant in complex with analogue nicotinamide (NIA) was determined. The NIA-complex showed a very similar structure to that of the N71S/F124W mutant protein. Slight changes in the side chains of Tyr⁶⁸, Phe⁷⁰, Phe¹²³ were observed and Trp¹²⁴ was found to be moved slightly(FIG. 7A). Hydrophobic stacking of the ring system of NIA, FMN and Trp¹²⁴ occurs in the active site of the N71S/F124W mutant. NIA stacked in parallel to the 5-membered ring of Trp¹²⁴ with an average distance of 3.7 Å.

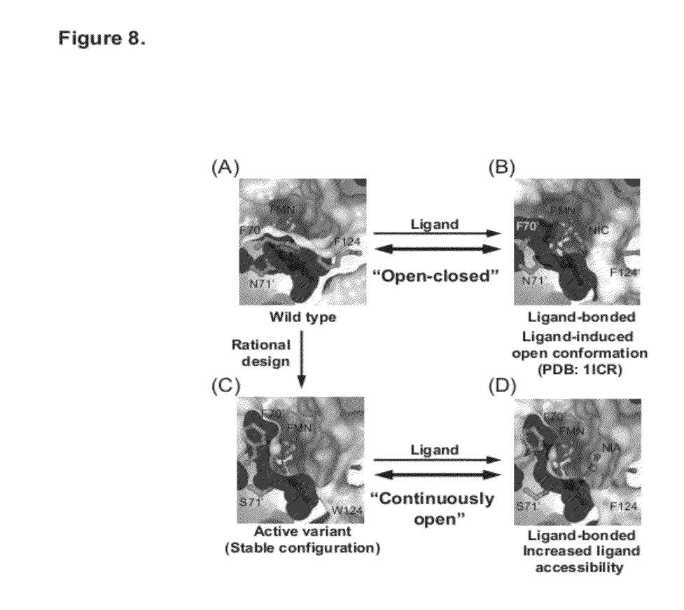

In the normal state, wild-type NfsB behaves in an “open-closed” manner. That is, the active site pocket becomes extended when a ligand binds to it(FIG. 8). The N71S/F124W mutant provides a more stable configuration of the pocket (FIG. 8C), suggesting that this mutant may behave in a “continuously open” manner to increase ligand accessibility (FIG. 8D).

### CONCLUSION

In sum, this study demonstrated the superior activity of a number of NfsB mutants (e.g., N71S/F124W) in converting NBDZ drugs such as FZ to non-toxic and inactive 7-amino metabolites both in vivo and in vitro. These mutants were designed based on the crystal structure of this enzyme as described herein. Certain mutants also showed a prolonged in vivo half-life. All these date indicate that the NfsB mutants described herein are effective antidotes for NBDZs.

### REFERENCES

1. Haefely, W. E. (1978) Br. J. Psychiatry 133, 231-238
2. Druid, H., Holmgren, P., and Ahlner, J. (2001) Forensic Sci. Int. 122, 136-141
3. Amir, L., and Waisman Y. (2006) Israeli J. Emerg. Med. 6(3), 62-66
4. Diehl, J. L., Guillibert, E., Guerot, E., Kimounn, E., and Labrousse, J. (2000) Ann. Med. Interne 151, A44-46
5. LinWu, S. W., Wang, A. H., and Peng, F. C. (2010) Expert Opin. Drug Metab. Toxicol. 6, 967-981
6. Jones, A. W., Kugelberg, F. C., Holmgren, A., and Ahlner, J. (2010) Forensic Sci. Int. doi:10.1016/j.forsciint.2010.06.015
7. Pirnay, S., Megarbane, B., Decleves, X., Risede, P., Borron, S. W., Bouchonnet, S., Perrin, B., Debray, M., Milan, N., Duarte, T., Ricordel, I., and Baud, F. J. (2008) Toxicol. Sci. 106, 64-73
8. Lai, S. H., and Teo, C. E. (2006) Ann. Acad. Med. Singapore 35, 508-511
9. Mizuno, K., Katoh, M., Okumura, H., Nakagawa, N., Negishi, T., Hashizume, T., Nakajima, M., and Yokoi, T. (2009) Drug Metab. Dispos. 37, 345-351
10. Peng, F. C., Chaing, H. H., Tang, S. H., Chen, P. C., and Lu, S. C. (2004) J. Toxicol. Environ. Health A 67, 109-124
11. Betten, D. P., Vohra, R. B., Cook, M. D., Matteucci, M. J., and Clark, R. F. (2006) J. Intensive Care Med. 21, 255-277
12. Spivey, W. H. (1992) Clin. Ther. 14, 292-305
13. LinWu, S. W., Syu, C. J., Chen, Y. L., Wang, A. H., and Peng, F. C. (2009) Biochem. Pharmacol. 78(1), 96-103
14. Orna, M. V., and Mason, R. P. (1989) J. Biol. Chem. 264, 12379-12384
15. Otwinowski, Z., and Minor, W. (1997) Methods Enzymol. 276, 307-326
16. Race, P. R., Lovering, A. L., Green, R. M., Ossor, A., White, S. A., Searle, P. F., Wrighton, C. J., and Hyde, E. I. (2005) J. Biol. Chem. 280, 13256-13264
17. Haynes, C. A., Koder, R. L., Miller, A. F., and Rodgers, D. W. (2002) J. Biol. Chem. 277, 11513-11520
18. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998) Acta Crystallogr. D Biol. Crystallogr. 54, 905-921
19. Parkinson, G. N., Skelly, J. V., and Neidle, S. (2000) J. Med. Chem. 43, 3624-3631
20. Kleywegt, G. J., and Jones, T. A. (1998) Acta Crystallogr. D Biol. Crystallogr. 54, 1119-1131
21. McRee, D. E. (1999) J. Struct. Biol. 125, 156-165
22. Olekhnovich, I. N., Goodwin, A., and Hoffman, P. S. (2009) FEBS J. 276, 3354-3364
23. Lemmer, B. (2007) Physiol. Behay. 90, 285-293
24. Kolosova, A. Y., Shim, W. B., Yang, Z. Y., Eremin, S. A., and Chung, D. H. (2006) Anal. Bioanal. Chem. 384, 286-294
25. Pavelka, A., Chovancova, E., and Damborsky, J. (2009) Nucleic Acids Res. 37, W376-383
26. Dundas, J., Ouyang, Z., Tseng, J., Binkowski, A., Turpaz, Y., and Liang, J. (2006) Nucleic Acids Res. 34, W116-118
27. Bogusz, M. J., Maier, R. D., Kruger, K. D., and Fruchtnicht, W. (1998) J. Chromatogr. 713, 361-369
28. Grove, J. I., Lovering, A. L., Guise, C., Race, P. R., Wrighton, C. J., White, S. A., Hyde, E. I., and Searle, P. F. (2003) Cancer Res. 63, 5532-5537
29. Lovering, A. L., Hyde, E. I., Searle, P. F., and White, S. A. (2001) J. Mol. Biol. 309, 203-213
30. Docampo, R., Mason, R. P., Mottley, C., and Muniz, R. P. (1981) J. Biol. Chem. 256, 10930-10933

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for detoxifying a nitrobenzodiazepine, the method comprising administering to a subject in need thereof an effective amount of a nitroreductase mutant, which, as compared to its wild-type counterpart, comprises a mutation at one or more positions corresponding to 40, 41, 68, 70, 71, and 124 in SEQ ID NO:1.

2. The method of claim 1, wherein the nitroreductase mutant is a mutant of E. coli nitroreductase NfsB.

3. The method of claim 2, wherein the nitroreductase mutant comprises amino acid residue G at position 40, L at position 41, G at position 68, H, W, or Y at position 70, S at position 71, H, K, N, W, or Y at position 124, or a combination thereof.

4. The method of claim 3, wherein the nitroductase mutant is a mutant selected from the group consisting of S40G, T41L, Y68G, F70H, F70W, F70Y, N71S, F124H, F124K, F124N, F124W, F124Y, and N71S/F124W.

5. The method of claim 1, wherein the subject is suffering from or suspected of having a side effect caused by a nitrobenzodiazepine.

6. The method of claim 5, wherein the nitrobenzodiazepine is selected from the group consisting of flunitrazepam, nitrazepam, cloazepam, nimetazepam, and meclonazepam.

7. The method of claim 1, wherein the nitroreductase mutant is co-administered with flumazenil.

8. The method of claim 7, wherein the nitroductase mutant is a mutant selected from the group consisting of S40G, T41L, Y68G, F70H, F70W, F70Y, N71S, F124H, F124K, F124N, F124W, F124Y, and N71S/F124W.

9. A method for converting a nitrobenzodiazepine to a 7-aminobenzodiazepine, the method comprising contacting the nitrobenzodiazepine with a nitroreductase mutant, which, as compared to its wild-type counterpart, comprises a mutation at one or more positions corresponding to 40, 41, 68, 70, 71, and 124 in SEQ ID NO:1.

10. The method of claim 9, wherein the nitrobenzodiazepine is selected from the group consisting of flunitrazepam, nitrazepam, cloazepam, nimetazepam, and meclonazepam.

11. The method of claim 9, wherein the nitroreductase mutant is a mutant of E. coli nitroreductase NfsB.

12. The method of claim 11, wherein the nitroreductase mutant comprises amino acid residue G at position 40, L at position 41, G at position 68, H, W, or Y at position 70, S at position 71, H, K, N, W, or Y at position 124, or a combination thereof.

13. The method of claim 12, wherein the nitroductase mutant is a mutant selected from the group consisting of S40G, T41L, Y68G, F70H, F70W, F70Y, N71S, F124H, F124K, F124N, F124W, F124Y, and N71S/F124W.

14. The method of claim 9, wherein the contacting step is performed by administering an effective amount of the nitroreductase mutant to a subject in need thereof.

15. A kit for detoxifying a nitrobenzodiazepine, comprising a nitrobenzodiazepine and a nitroreductase mutant, wherein the nitroreductase mutant comprises a mutation at one or more positions corresponding to 40, 41, 68, 70, 71, and 124 in SEQ ID NO:1 as compared to its wild-type counterpart.

16. The kit of claim 15, further comprising flumazenil.


SEQUENCE LISTING (1 sequence)

SEQ ID NO: 1 - protein, 217 aa, E. coli
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV